SEQUENCE LISTING (31 sequences)

SEQ ID NO: 1 - DNA, 20 bp, synthetic construct
  source 1..20: mol_type = other DNA
cttggctgctgctcttatcc

SEQ ID NO: 2 - DNA, 16408 bp, synthetic construct
  source 1..16408: mol_type = other DNA
taaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcggcgaacaaagcaccagtggtctagtggtagaatagtaccctgccacggtacagacccgggttcgattcccggctggtgcacttggctgctgctcttatccgtttcagagctatgctggaaacagcatagcaagttgaaataaggctagtccgttatcaacttgaaaaagtggcaccgagtcggtgcaacaaagcaccagtggtctagtggtagaatagtaccctgccacggtacagacccgggttcgattcccggctggtgcattttttttttcgttttgcattgagttttctccgtcgcatgtttgcagttttattttccgttttgcattgaaatttctccgtctcatgtttgcagcgtgttcaaaaagtacgcagctgtatttcacttatttacggcgccacattttcatgccgtttgtgccaactatcccgagctagtgaatacagcttggcttcacacaacactggtgacccgctgacctgctcgtacctcgtaccgtcgtacggcacagcatttggaattaaagggtgtgatcgatactgcttgctgctaagcttgcatgcttaattaaactagtctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagccctaggcctactagatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgagctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaa

SEQ ID NO: 3 - DNA, 16402 bp, synthetic construct
  source 1..16402: mol_type = other DNA
taaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcggcgaacaaagcaccagtggtctagtggtagaatagtaccctgccacggtacagacccgggttcgattcccggctggtgcaggagaccggtctcggtttcagagctatgctggaaacagcatagcaagttgaaataaggctagtccgttatcaacttgaaaaagtggcaccgagtcggtgcaacaaagcaccagtggtctagtggtagaatagtaccctgccacggtacagacccgggttcgattcccggctggtgcattttttttttcgttttgcattgagttttctccgtcgcatgtttgcagttttattttccgttttgcattgaaatttctccgtctcatgtttgcagcgtgttcaaaaagtacgcagctgtatttcacttatttacggcgccacattttcatgccgtttgtgccaactatcccgagctagtgaatacagcttggcttcacacaacactggtgacccgctgacctgctcgtacctcgtaccgtcgtacggcacagcatttggaattaaagggtgtgatcgatactgcttgctgctaagcttgcatgcttaattaaactagtctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagccctaggcctactagatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgagctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaa

SEQ ID NO: 4 - DNA, 20 bp, synthetic construct
  source 1..20: mol_type = other DNA
ggagaagttgggtagtacct

SEQ ID NO: 5 - DNA, 20 bp, synthetic construct
  source 1..20: mol_type = other DNA
ctccgcaacaattaggacaa

SEQ ID NO: 6 - DNA, 20 bp, synthetic construct
  source 1..20: mol_type = other DNA
ctggctgctgcggagaagtt

SEQ ID NO: 7 - DNA, 20 bp, synthetic construct
  source 1..20: mol_type = other DNA
tgctcttatcctggctgctg

SEQ ID NO: 8 - DNA, 20 bp, synthetic construct
  source 1..20: mol_type = other DNA
ccaacttctccgcagcagcc

SEQ ID NO: 9 - DNA, 20 bp, synthetic construct
  source 1..20: mol_type = other DNA
ctacccaacttctccgcagc

SEQ ID NO: 10 - DNA, 20 bp, synthetic construct
  source 1..20: mol_type = other DNA
tatcctggctgctgcggaga

SEQ ID NO: 11 - DNA, 20 bp, synthetic construct
  source 1..20: mol_type = other DNA
tccgcagcagccaggataag

SEQ ID NO: 12 - DNA, 20 bp, synthetic construct
  source 1..20: mol_type = other DNA
gcagcagccaggataagagc

SEQ ID NO: 13 - DNA, 20 bp, synthetic construct
  source 1..20: mol_type = other DNA
aggtactacccaacttctcc

SEQ ID NO: 14 - DNA, 20 bp, synthetic construct
  source 1..20: mol_type = other DNA
caacttctccgcagcagcca

SEQ ID NO: 15 - DNA, 20 bp, synthetic construct
  source 1..20: mol_type = other DNA
cgcagcagccaggataagag

SEQ ID NO: 16 - DNA, 20 bp, synthetic construct
  source 1..20: mol_type = other DNA
ccgcagcagccaggataaga

SEQ ID NO: 17 - DNA, 20 bp, synthetic construct
  source 1..20: mol_type = other DNA
tcttatcctggctgctgcgg

SEQ ID NO: 18 - DNA, 20 bp, synthetic construct
  source 1..20: mol_type = other DNA
gataagagcagcagccaaga

SEQ ID NO: 19 - DNA, 77 bp, synthetic construct
  source 1..77: mol_type = other DNA
aacaaagcaccagtggtctagtggtagaatagtaccctgccacggtacagacccgggttcgattcccggctggtgca

SEQ ID NO: 20 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 21 - DNA, 86 bp, synthetic construct
  source 1..86: mol_type = other DNA
gtttcagagctatgctggaaacagcatagcaagttgaaataaggctagtccgttatcaacttgaaaaagtggcaccgagtcggtgc

SEQ ID NO: 22 - DNA, 41 bp, synthetic construct
  source 1..41: mol_type = other DNA
ggagtgagtacggtgtgcacaattaggacaagggcaaccaa

SEQ ID NO: 23 - DNA, 37 bp, synthetic construct
  source 1..37: mol_type = other DNA
gagttggatgctggatggctggctgctgaccttgctc

SEQ ID NO: 24 - DNA, 2493 bp, Triticum aestivum
  source 1..2493: mol_type = genomic DNA
atgactaagcggttggttctttttgcggcggtagtcgtcgcccttgtggctctcaccgctgctgaaggtgaggcctctgggcaactacagtgtgagcgcgagctccaggagcactcgcttaaggcatgccgacaggtcgtagaccagcagctccgagacgttagccccgagtgccaacccgtcggcggcggcccggtcgcgagacaatatgagcagcaagtcgtggtgccgcccaagggtggatctttctaccccggcgagaccacgccaccacagcaactccaacaaagtatactttggggaatacctgcactactaagaaggtattacctaagtgtaacttctccgcaacgggtttcatactatccaggccaagcttcttcgcaacggccaggacaaggtcagcagccaggacaaggacaacaagaatactacctaacttctccgcaacagtcaggacaatggcaacaaccgggacaagggcaatcagggtactacccaacttctccgcagcagtcaggacaagagcaaccagggtactatccaacttctccatggcagccagaacaattgcaacaaccaacacaagagcaacaaagacagcaaccaggacaagggcagcaactaagacaaggacaacaaggtcagcagtcaggacaagggcaaccaagatactatccaacttcttcgcagcagccaggacaattgcaacaactaacacaaggccaacaagggcagcaaccagaacgagggcaacaaggccaacagtcaggacaagggcaacaactaggacaagggcaacaaggtcagcagccaggacaaaagcaacaatcaggacaaggacaacaagggtactacccaatttctccgcaacagttaggacaagggcaacagtcaggacaagggcaactagggtactacccaacttctccgcagcagtcaggacaaggacaatcaggatactacccaacttctgcgcagcagccaggacaattgcaacaatcaacacaagagcagcaattaggacaagagcaacaagatcagcaatcaggacaagggcgacaaggtcaacagtcaggacaaaggcaacaagatcagcagtcaggacaagggcagcaaccgggacaaaggcagccagggtactactcatcttctccgcaacaattaggacaagggcaaccaaggtactacccaacttctccgcagcagccaggataagagcagcagccaagacaattgcaacaaccagaacaagggcaacaaggtcagcagccagaacaagggcagcaaggtcagcagccaggacaaggggagcaaggtcagcagccaggacaagggcaacaagggcaacaaccgggacaagggcagccagggtactacccaacttctccgcagcagtcgggacaagggcaaccagggtactacccaacttctccacagcagtcaggacaattgcaacaaccagcacaagggcagcaaccaggacaagagcaacaaggtcaacagccaggacaagggcaacaaggtcaacagccaggacaagggcagcaaccgggacaagggcagccagggtactacccaacttctccgcagcagtcaggacaagagcaacagctagaacaatggcaacagtcaggacaggggcaaccagggcactacccaacttctccgttgcagccaggacaagggcaaccagggtactacccaacttctccacaacagataggacaagggcagcagccaggacaattgcaacaaccaacacaagggcaacaagggcagcaaccaggacaagggcaacaaggtcaacagccaggacaagggcaacaaggtcagcagccaggacaagggcagcaaccaggacaagggcagccagggtactacccaacttctttgcagcagtcaggacaagggcaacagccaggacaatggcaacaaccaggacaaggacaaccagggtactacccaacttcttcgttgcagccagaacaagggcaacaagggtactacccaacttctcagcagcaaccaggacaagggccgcaaccaggacaatggcaacaatcaggacaagggcaacaagggtactacccaacttctccgcagcagtcaggacaagggcaacagccaggacaatggctgcaaccaggacaatggctgcaatcagggtactacctaacttctccgcagcagttaggacaagggcaacagccaagacaatggctgcaaccaagacaagggcaacaaggatactacccaacttctccgcagcagtcaggacaagggcaacaattaggacaagggcaacaaggatactacccaacttctccgcagcagtcaggacaagggcaacaaggctacgacagcccataccatgttagcgcggagcaccatgcggccagcctaaaggtggcaaaggcacagcagctcgcggcacagctgccggcaatgtgccggctagagggcggcgacgcattgttggccagccagtga

SEQ ID NO: 25 - DNA, 17 bp, synthetic construct
  source 1..17: mol_type = other DNA
gtaaaacgacggccagt

SEQ ID NO: 26 - DNA, 18 bp, synthetic construct
  source 1..18: mol_type = other DNA
tgcactgcaggcatgcaa

SEQ ID NO: 27 - DNA, 527 bp, synthetic construct
  source 1..527: mol_type = other DNA
catgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcggc

SEQ ID NO: 28 - DNA, 291 bp, synthetic construct
  source 1..291: mol_type = other DNA
ttttttttttcgttttgcattgagttttctccgtcgcatgtttgcagttttattttccgttttgcattgaaatttctccgtctcatgtttgcagcgtgttcaaaaagtacgcagctgtatttcacttatttacggcgccacattttcatgccgtttgtgccaactatcccgagctagtgaatacagcttggcttcacacaacactggtgacccgctgacctgctcgtacctcgtaccgtcgtacggcacagcatttggaattaaagggtgtgatcgatactgcttgctgct

SEQ ID NO: 29 - DNA, 17340 bp, synthetic construct
  source 1..17340: mol_type = other DNA
taaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttagtaattcatccaggtcaccaagttctaggattttcagaactgcaacttattttatcaaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcgtgagaccaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccggtctcggttttagagctagaaatagcaagttaaaataaggctagtccgttatcaacttgaaaaagtggcaccgagtcggtgcttttttttttcgttttgcattgagttttctccgtcgcatgtttgcagttttattttccgttttgcattgaaatttctccgtctcatgtttgcagcgtgttcaaaaagtacgcagctgtatttcacttatttacggcgccacattttcatgccgtttgtgccaactatcccgagctagtgaatacagcttggcttcacacaacactggtgacccgctgacctgctcgtacctcgtaccgtcgtacggcacagcatttggaattaaagggtgtgatcgatactgcttgctgctaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagccctaggcctactagatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgagctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaa

SEQ ID NO: 30 - DNA, 24 bp, synthetic construct
  source 1..24: mol_type = other DNA
tgcacttggctgctgctcttatcc

SEQ ID NO: 31 - DNA, 24 bp, synthetic construct
  source 1..24: mol_type = other DNA
aaacggataagagcagcagccaag

(19) United States
(12) Patent Application Publication
(10) Pub. No.: US 2025/0122521 A1
(43) Pub. Date: Apr. 17, 2025

(54) Method for Activating Expression of Silenced Glu-1Ax-null Subunit in Wheat and Related Biomaterials

(71) Applicant: CROP RESEARCH INSTITUTE, SHANDONG ACADEMY OF AGRICULTURAL SCIENCES, JINAN (CN)

(72) Inventors: Jihu LI, JINAN (CN); Genying LI, JINAN (CN); Yulian LI, JINAN (CN); Jie GAO, JINAN (CN); Guoqi SONG, JINAN (CN); Shujuan ZHANG, JINAN (CN); Rongzhi ZHANG, JINAN (CN); Wei LI, JINAN (CN); Yanxiang LIN, JINAN (CN)

(21) Appl. No.: 18/538,183

(22) Filed: Dec. 13, 2023

(30) Foreign Application Priority Data
May 31, 2023 (CN) CN202310639011.3

(51) Int. Cl. C12N 15/82; C12N 9/22; C12N 15/11; C12Q 1/6869; C12Q 1/6895

(52) U.S. Cl. CPC C12N 15/8262; C12N 9/22; C12N 15/11; C12N 15/8205; C12Q 1/6869; C12Q 1/6895; C12N 2310/20; C12N 2800/80; C12Q 2600/13

(57) ABSTRACT

The present invention provides a method for activating expression of a Glu-1Ax-null subunit in wheat by gene editing, the method comprising: designing a specific sgRNA for targeting a premature termination codon in a Glu-1Ax-null gene of wheat; cloning the specific sgRNA into a pair of BsaI sites of a vector pEtRNA to construct a CRISPR/SpCas9 editing vector for the Glu-1Ax-null gene; and transforming the CRISPR/SpCas9 editing vector for the Glu-1Ax-null gene into wheat cell via Agrobacterium-mediated transformation and selecting a homozygous line in which the premature termination codon is disrupted. The inventive specific sgRNA is designed near the 1216-1218-site premature termination codon of the Glu-1Ax-null gene, the CRISPR/SpCas9 editing vector is transformed into wheat via Agrobacterium-mediated transformation, and the premature termination codon can be specially disrupted, thereby activating expression of silenced high-molecular-weight glutenin Glu-1Ax-null subunit in wheat.

### RELATED APPLICATION

The application claims the benefit of CN202310639011.3 filed May 31, 2023, which is incorporated herein by reference in its entirety.

### INCORPORATION OF A SEQUENCE LISTING

A Sequence Listing is provided herewith as a Sequence Listing XML, “Sequence Listing-EIC2023021001CN-US” created on Aug. 12, 2023 and then amended on Dec. 30, 2024 to have a size of 82.6 KB (measured in MS-Windows 11®). The amended Sequence Listing, electronically filed herewith, does not extend beyond the scope of the specification and the drawings originally filed on Dec. 13, 2023 and thus does not give rise to new matter. The contents of the Sequence Listing XML are incorporated by reference herein in their entirety.

### FIELD OF THE INVENTION

The present invention belongs to the field of biotechnology. More particularly, the present invention relates to a method for activating expression of a high-molecular-weight glutenin Glu-1Ax-null subunit in wheat and related biomaterials.

### BACKGROUND OF THE INVENTION

Wheat (Triticum aestivum L.) is the second largest food crop, and the sustainable development of its production is very important for ensuring domestic food security. Wheat flour can be used to make a variety of foods such as breads, steamed breads, noodles, pastries, etc. These processing properties mainly depend on composition and content of seed storage proteins (SSP) [Shewry et al. (2002)]. SSP mainly comprise glutenins and gliadins. Glutenins determine strength and elasticity of dough, and gliadins mainly affect the viscosity of dough [Veraverbeke and Delcour (2002)]. According to protein molecular weight difference, glutenins comprise high-molecular-weight glutenin subunits (HMW-GS) and low-molecular-weight glutenin subunits (LMW-GS) [Shewry et al. (1995)]. Although accounting for only about 10% of the content of SSP, HMW-GS constitute dough skeleton and determine strength and elasticity of dough, and the variation on its composition and content can explain 45-70% of the processing quality variations [Branlard and Dardevet (1985); Halford et al. (1992); He et al. (2005); Payne et al. (1987)].

HMW-GS are encoded by the Glu-1 gene on the long arm of homocologous group 1 chromosomes of wheat, and are specifically expressed in endosperm at seed development middle-later stage. They are encoded by three loci (Glu-1A, Glu-1B and Glu-1D) located on the long arm of the homocologous group 1 chromosomes, and each locus contains two closely linked genes, which are x-type subunit encoding larger molecular weight and y-type subunit encoding smaller molecular weight. Thus, in theory, 6 HMW-GS are expressed by per common wheat variety, but only 3-5 HMW-GS are expressed due to gene silencing effect. Increasing the number of expressed HMW-GS is an important way to improve wheat processing quality [Halford et al. (1992); Vawser et al. (2004)].

At present, the expressed HMW-GS are transformed into a wheat variety by breeding experts mainly through crossing to increasing the number of expressed HMW-GS. However, these traditional breeding methods are often accompanied by linkage drag, seriously affecting other agronomic traits of the variety, and often require multi-time back-crossing, resulting in long breeding period, high cost and low efficiency.

Gene editing technique is an advanced bio-technique for accurate modification (knocking-out, insertion and substitution) of endogenous gene of organism, and has the advantages of simplicity, high efficiency and short test period. The third-generation gene editing technique represented by CRISPR/SpCas9 system has been widely applied in gene function study and trait genetic improvement of crops [Zhan et al. (2020)].

In view of the fact that most wheat varieties currently used in agricultural production cannot express all HMW-GS, the inventor of the present invention focuses on increasing the number of expressed HMW-GS in wheat grain by means of molecular biology and genetic engineering to develop a new way for improving processing quality of wheat. This work has great significance to wheat breeding.

### OBJECTS AND SUMMARY OF THE INVENTION

Therefore, it is an object of the claimed invention to provide precise editing of a Glu-1Ax-null gene of wheat (silenced high-molecular-weight glutenin Glu-1Ax subunit encoding gene) by gene editing technique to activate expression of silenced Glu-1Ax-null subunit in wheat, increase the number of expressed HMW-GS and improve wheat processing quality.

For ease of understanding, some terms in the present invention are defined or explained below.

In the present invention, the term “silenced Glu-1Ax subunit”, “Glu-1Ax-null subunit”, “Glu-1Ax-null gene” or “Glu-1Ax-null” are used to represent: the subunits and their coding genes incapable of expressing HMW-GS in x-type subunits encoding the Glu-1A site of the HMW-GS. According to the research of the present inventor, the incapability of these subunits on expressing HMW-GS is attributed to the presence of a premature termination codon (TAA).

In the present invention, the term “vector pEtRNA” refers to a gene editing vector of the CRISPR/SpCas9 system used in the present invention for precise editing of the Glu-1Ax-null gene and obtained by modification based on the known “vector pBUE411”. The modification includes optimization of an sgRNA scaffold sequence and introduction of tRNA-mediated sgRNA release manner. The vector pBUE411 has sequence shown as SEQ ID NO. 29, and the vector pEtRNA has sequence shown as SEQ ID NO. 3.

In the present invention, the term “sgRNA scaffold sequence” is a sequence located in an sgRNA expression cassette of the gene editing vector of the CRISPR/SpCas9 system and bound with SpCas9 protein, excluding the sgRNA sequence. The term “non-optimized sgRNA scaffold sequence” refers to the original sgRNA scaffold sequence in the “vector pBUE411”, and has 5′-3′ sequence shown as SEQ ID NO.20 and a length of 76 bp. The term “optimized sgRNA scaffold sequence” refers to the sgRNA scaffold sequence in the vector pEtRNA modified based on the vector pBUE411, and has 5′-3′ sequence shown as SEQ ID NO. 21 and a length of 86 bp. The optimization improves the gene editing efficiency of activating wheat Glu-1Ax-null subunit. For simplicity, “MSS sequence” used in the application refers to the “optimized sgRNA scaffold sequence”.

In the present invention, the term “conventional sgRNA release manner” is relative to “tRNA-mediated sgRNA release manner” proposed in the present invention. The “conventional sgRNA release manner” refers to the original sgRNA release manner in the vector pBUE411. The “tRNA-mediated sgRNA release manner” refers to that: in the vector pEtRNA obtained by modifying the vector pBUE411 in the present invention, the upstream and the downstream of “sgRNA+sgRNA scaffold sequence” are respectively introduced with tRNA sequences, thereby modifying the original sgRNA release manner into tRNA-mediated sgRNA release manner.

Through gene sequencing, the inventor of the present invention finds that, compared with the expressed Glu-1Ax gene, the unexpressed Glu-1Ax-null gene has a C-to-T single base substitution at the 1216 site in the coding region, and the corresponding trivalent codon composed of the 1216-site, 1217-site and 1218-site bases is changed from glutamine (CAA) to the premature termination codon (TAA), which may lead to premature termination of protein translation. That may be the cause of non-expression of the Glu-1Ax-null subunit.

In order to make the premature termination codon have a frameshift mutation or delete the premature termination codon and activate expression of the Glu-1 Ax-null subunit, the inventor of the present invention makes a large number of experimental attempts, and finally accidentally designs a specific single-stranded guide RNA (sgRNA) suitable for CRISPR/SpCas9 editing system. A gene editing vector with the function of deleting the premature termination codon is constructed and transformed into common wheat to achieve accurate deletion of the premature termination codon, thereby activating the expression of Glu-1Ax-null subunit of high-molecular-weight glutenin, increasing the number of expressed HMW-GS and improving wheat processing quality.

Based on the above findings, the first aspect of the present invention is to provide a method for activating expression of a Glu-1 Ax-null subunit in wheat by gene editing, comprising the steps of:
designing a specific sgRNA for targeting a premature termination codon in a Glu-1Ax-null gene of wheat;
cloning the specific sgRNA onto a pair of BsaI sites of a vector pEtRNA to construct a CRISPR/SpCas9 editing vector for the Glu-1Ax-null gene, wherein a sequence of the vector pEtRNA is shown as SEQ ID NO. 3; and
transforming the CRISPR/SpCas9 editing vector for the Glu-1Ax-null gene into wheat cell via Agrobacterium-mediated transformation and selecting a homozygous line in which the premature termination codon is disrupted.

The second aspect of the present invention is to provide a specific sgRNA for targeting a premature termination codon in a Glu-1Ax-null gene of wheat, wherein: a target sequence of the specific sgRNA corresponds to a sequence having length of 20-22 nucleotides between 1208-1235 sites of the Glu-1Ax-null gene.

In some preferable embodiments of the present invention, a nucleotide sequence of the specific sgRNA is shown as SEQ ID NO. 1, i.e., 5′-cttggctgctgctcttatcc-3′.

The third aspect of the present invention is to provide a CRISPR/SpCas9 editing vector for a Glu-1Ax-null gene of wheat, comprising: a U3-sgRNA expression cassette regulated by a wheat TaU3 promoter and an SpCas9 expression cassette regulated by a ZmUbi promoter, wherein: the U3-sgRNA expression cassette includes, from upstream to downstream: the wheat TaU3 promoter, a first tRNA, a specific sgRNA for targeting the premature termination codon in the Glu-1Ax-null gene of wheat, an MSS sequence, a second tRNA which is identical to first tRNA, and an OsU3 terminator.

In some preferable embodiments of the present invention, the nucleotide sequence of the CRISPR/SpCas9 editing vector for the Glu-1Ax-null gene is shown as SEQ ID NO. 2.

The specific sgRNA in the present invention is designed near the premature termination codon at 1216-1218 sites of the Glu-1Ax-null gene, and the CRISPR/SpCas9 editing vector is transformed into wheat via Agrobacterium-mediated transformation to specifically disrupt the premature termination codon, thereby activating expression of silenced Glu-1Ax-null subunit in wheat.

### BRIEF DESCRIPTION OF FIGURES

The implementation scheme of the present invention is described in detail in combination with the figures, wherein:

FIG. 1 is a schematic diagram of the target sequence of the sgRNA as set forth in SEQ ID NO. 32 in Example 1;

FIG. 2 is a schematic diagram of the gene editing vector constructed in Example 1 as set forth in SEQ ID NO. 2, wherein: the diamond shape represents the sgRNA as set forth in SEQ ID NO. 1; the square shape at its downstream represents the sgRNA scaffold sequence, i.e., the MSS sequence as set forth in SEQ ID NO. 21; and the tRNA is set forth in SEQ ID NO. 19;

FIG. 3 shows genotypes of the three lines with 3bp sequence deleted in the T0-generation transgenic lines in Example 2, wherein: WT represents the wild type as set forth in SEQ ID NO. 33; the first rows in L4, L6 and L7 indicate the wild type as set forth in SEQ ID No. 33; the second rows in L4, L6 and L7 indicate the sequence of the genotypes as set forth in SEQ ID NO. 34 in which the premature termination codon TAA is deleted;

FIG. 4 shows the sequencing chromatograms of sgRNA region T4-generation gene-edited homozygous lines in Example 2, wherein: WT represents the wild type as set forth in SEQ ID NO. 33; and the proportion of-3bp genotype in a single plant reaches 100% in L4-2, L6-5 and L7-4 lines as set forth in SEQ ID NO. 34 in which the premature termination codon TAA is deleted;

FIG. 5 is protein band results of seed HMW-GS composition analyzed by SDS-PAGE in Example 3; and

FIG. 6 is analysis results of flour processing qualities of gene-edited lines in Example 4, wherein ZSV, GI, ST and FQN represent flour settlement value, gluten index, stabilization time and flour quality number, respectively; different letters indicate significant differences (P<0.05), and data are expressed as means±standard error (n=3).

### DETAILED DESCRIPTION OF THE EMBODIMENTS

The following is a further detailed description of the present invention in combination with implementations. The given examples are intended only to explain the present invention, not to limit the scope of the present invention.

### Gene Editing Method

The first aspect of the present invention is to provide a method for activating expression of a Glu-1Ax-null subunit in wheat with gene editing, comprising the steps of:
designing a specific sgRNA for targeting a premature termination codon in a Glu-1Ax-null gene of wheat or the upstream sequence of the premature termination codon;
cloning the specific sgRNA onto a pair of BsaI sites of a vector pEtRNA to construct a CRISPR/SpCas9 editing vector for the Glu-1Ax-null gene;
transforming the CRISPR/SpCas9 editing vector for the Glu-1Ax-null gene into wheat cell via Agrobacterium-mediated transformation and selecting a homozygous line in which the premature termination codon is disrupted.

Specifically, in some preferable embodiments of the present invention, a method for activating expression of a Glu-1Ax-null subunit in wheat includes the following steps:
transforming a recombinant vector containing the specific sgRNA and an SpCas9 expression element into Escherichia coli, screening the Escherichia coli for positive clones; and verifying the positive clones through sequencing;
transforming a vector of the Escherichia coli into Agrobacterium through chemical transformation; using PCR to verify a positive Agrobacterium clone; and introducing the positive Agrobacterium clone into wheat to obtain transgenic wheat; and
screening the transgenic wheat for a gene-edited plant in which the premature termination codon is disrupted; self-crossing the plant to obtain a progeny; and selecting the homozygous line from the progeny in which the premature termination codon is disrupted.

According to the method for activating expression of a Glu-1Ax-null subunit in wheat in the present invention, a target sequence of the specific sgRNA corresponds to a sequence having length of 20-22 nucleotides between 1208-1235 sites of the Glu-1Ax-null gene.

In some preferable embodiments of the present invention, a nucleotide sequence of the specific sgRNA is shown as SEQ ID NO. 1, i.e., 5′-cttggctgctgctcttatcc-3′.

According to the method for activating expression of a Glu-1Ax-null subunit in wheat in the present invention, the vector pEtRNA is obtained by modification based on the vector pBUE411. The modification includes optimization of an sgRNA scaffold sequence and introduction of tRNA-mediated sgRNA release manner. In some embodiments of the present invention, the vector pEtRNA adopted in the gene editing method of the present invention has sequence shown as SEQ ID NO. 3.

In some preferable embodiments of the present invention, the CRISPR/SpCas9 editing vector comprises: a U3-sgRNA expression cassette regulated by a wheat TaU3 promoter and an SpCas9 expression cassette regulated by a ZmUbi promoter, wherein: the U3-sgRNA expression cassette includes, from upstream to downstream: the wheat TaU3 promoter, a first tRNA, a specific sgRNA for targeting a premature termination codon in a Glu-1Ax-null gene of wheat, an MSS sequence, a second tRNA which is identical to first tRNA, and an OsU3 terminator.

The MSS sequence is shown as SEQ ID NO. 21.

In some embodiments of the present invention, the first tRNA is shown as SEQ ID NO. 19, and the second tRNA is shown as SEQ ID NO. 19.

In some preferable embodiments of the present invention, a nucleotide sequence of the CRISPR/SpCas9 editing vector for the Glu-1Ax-null gene is shown as SEQ ID NO. 2.

sgRNA

The second aspect of the present invention is to provide a specific sgRNA for targeting a premature termination codon in a Glu-1Ax-null gene of wheat, wherein: a target sequence of the specific sgRNA corresponds to a sequence having length of 20-22 nucleotides between 1208-1235 sites of the Glu-1Ax-null gene.

In some preferable embodiments of the present invention, a nucleotide sequence of the specific sgRNA is shown as SEQ ID NO. 1, i.e., sgRNA 5′-cttggctgctgctcttatcc-3′.

The inventor of the present invention designs a large number of target sequences, corresponding to the sequence near the premature termination codon of Glu-1Ax-null gene, as sgRNA for editing a Glu-1Ax-null gene of wheat, such as the 5′-3′ sequences used in the comparative examples of the application as follows: ggagaagttgggtagtacct (SEQ ID NO. 4), ctccgcaacaattaggacaa (SEQ ID NO. 5), aggtactacccaacttetcc (SEQ ID NO. 13), tgctcttatcctggctgctg (SEQ ID NO. 7), ctggctgctgcggagaagtt (SEQ ID NO. 6), ccaacttctccgcagcagcc (SEQ ID NO. 8), tatcctggctgctgcggaga (SEQ ID NO. 10), etc. The results show that only the sequence targeting 1208-1235 sites of the Glu-1Ax-null gene has the required editing function, namely: creating a mutation to the premature termination codon, causing a frameshift mutation and disrupting the premature termination codon, wherein the editing result with the sequence cttggctgctgctcttatcc (SEQ ID NO. 1) as sgRNA is the optimal.

### CRISPR/SpCas9 Editing Vector

The third aspect of the present invention is to provide a CRISPR/SpCas9 editing vector for a Glu-1Ax-null gene of wheat, comprising: a U3-sgRNA expression cassette regulated by a wheat TaU3 promoter and an SpCas9 expression cassette regulated by a ZmUbi promoter, wherein: the U3-sgRNA expression cassette includes, from upstream to downstream: the wheat TaU3 promoter, a first tRNA, a specific sgRNA for targeting a premature termination codon in a Glu-1Ax-null gene of wheat, an MSS sequence, a second tRNA which is identical to first tRNA, and an OsU3 terminator.

The MSS sequence is shown as SEQ ID NO. 21.

The CRISPR/SpCas9 editing vector for the Glu-1Ax-null gene provided by the present invention is a gene editing vector obtained by cloning the sgRNA onto a pair of BsaI sites of a vector pEtRNA.

The vector pEtRNA is obtained by modification based on the “vector pBUE411”. The modification includes optimization of an sgRNA scaffold sequence and introduction of tRNA-mediated sgRNA release manner. In some embodiments of the present invention, the vector pEtRNA adopted in the gene editing method of the present invention has sequence shown as SEQ ID NO. 3.

Specifically, the modification includes the following steps:
(1) Synthesizing an sgRNA expression cassette required by vector pEtRNA in the 5′-3′ direction: a first HindIII restriction site, a TaU3 promoter, a first tRNA, a first BsaI restriction site, a second BsaI restriction site, an MSS sequence, a second tRNA, an OsU3 terminator sequence and a second HindIII restriction site.
(2) Respectively digesting the vector pBUE411 and the synthesized sgRNA expression cassette sequence with HindIII endonuclease, respectively extracting the digested vector and the synthesized sequence by an agarose gel extraction kit, ligating the digested vector and the synthesized sequence overnight by T4 ligase, and transforming the ligated product into Escherichia coli.
(3) Verifying whether the synthetic sequence is successfully cloned into the vector pBUE411 by sequencing with a primer pTaU3, wherein the vector with positive clone is the vector pEtRNA employed in the present invention, and the pTaU3 comprises a forward primer sequence (5′-3′): GTAAAACGACGGCCAGT (SEQ ID NO. 25) and a reverse primer sequence (5′-3′): TGCACTGCAGGCATGCAA (SEQ ID NO. 26).

The sequence of the TaU3 promoter is shown as SEQ ID NO. 27. The sequence of the OsU3 terminator is shown as SEQ ID NO. 28.

Through a large number of experimental studies, the inventor of the present invention unexpectedly finds that, the combination of the sgRNA selected in the present invention, the optimized sgRNA scaffold (MSS sequence) and the tRNA-mediated sgRNA release manner can precisely edit a Glu-1Ax-null gene of wheat and disrupt the premature termination codon, thereby activating expression of silenced Glu-1Ax-null subunit in wheat, increasing the number of expressed HMW-GS and improving wheat processing quality.

In the CRISPR/SpCas9 editing vector for the Glu-1Ax-null gene of wheat provided by the present invention, a target sequence of the specific sgRNA corresponds to a sequence having length of 20-22 nucleotides between 1208-1235 sites of the Glu-1Ax-null gene.

In some preferable embodiments of the present invention, a nucleotide sequence of the specific sgRNA is shown as SEQ ID NO. 1, i.e., 5′-cttggctgctgctcttatcc-3′.

In the CRISPR/SpCas9 editing vector for a Glu-1Ax-null gene of wheat provided by the present invention, the first or second tRNA is a fragment of sequence containing 77 nucleotides, and comprises a recognition restriction site for RnaseP at its 5′-terminal and a recognition restriction site for RnazeZ at its 3′-terminal after transcription. The two sites of the first or second tRNA sequence can be digested by plant endogenous nuclease to release sgRNA.

In some preferable embodiments of the present invention, the nucleotide sequence of the first tRNA is shown as SEQ ID NO. 19, and a nucleotide sequence of the second tRNA is shown as SEQ ID NO. 19.

The inventor of the present invention tries a variety of vectors and sgRNA release manners, and finally determines that the combination of the inventive optimized sgRNA scaffold (MSS sequence) with tRNA-mediated sgRNA release manner can successfully edit a Glu-1Ax-null gene of wheat and disrupt the premature termination codon.

In some preferable embodiments of the present invention, the nucleotide sequence of the CRISPR/SpCas9 editing vector for the Glu-1Ax-null gene is shown as SEQ ID NO. 2.

### EXAMPLES

### Plant Materials and Experimental Reagents

The wheat gene editing receptor employed in the following examples is the spring wheat variety Fielder. The sequence of a Glu-1Ax-null gene of the Fielder is shown as SEQ ID NO. 24. The restriction endonuclease BsaI required for gene editing vector construction is purchased from New England Biolabs (Beijing) Ltd., and both T4 ligase and agarose gel extraction kit are purchased from Thermo Fisher Scientific (China) Co., Ld. Escherichia coli competent cells DH5α are purchased from TransGen Biotech Co., Ltd., and Agrobacterium competent cells EHA 105 are purchased from Beijing Huayueyang Biotech Co., Ltd. Escherichia coli plasmid small extraction kit and 2×Accurate Taq Master Mix are purchased from Accurate Biology Co., Ltd. Other chemical reagents are purchased from Sinopharm Chemical Reagent Co., Ltd. The primers used in the experiments are synthesized by Beijing Tsingke Biotechnology Co., Ltd.

A method for preparing gene editing vector pEtRNA includes: synthesizing a sgRNA expression cassette required for vector pEtRNA in Beijing Tsingke Biotechnology Co., Ltd. and including an HindIII restriction site, TaU3 promoter, a first tRNA, a first BsaI restriction site, a second BsaI restriction site, an MSS sequence, a second tRNA, an OsU3 terminator sequence and a second HindIII restriction site in 5′-3′ direction; respectively digesting the vector pBUE411 and the synthesized sgRNA expression cassette sequence with HindIII endonuclease, respectively extracting the digested vector and the synthesized sequence by an agarose gel extraction kit, ligating the digested vector and the synthesized sequence overnight by T4 ligase, and transforming the ligated product into Escherichia coli; verifying whether the synthetic sequence is successfully cloned into the vector pBUE411 by sequencing with a primer pTaU3 (F: GTAAAACGACGGCCAGT, SEQ ID NO. 25; R: TGCACTGCAGGCATGCAA, SEQ ID NO. 26), wherein the vector with positive clone is the vector pEtRNA employed in the present invention, the sequence of the TaU3 promoter is shown as SEQ ID NO. 27, and the sequence of the OsU3 terminator is shown as SEQ ID NO. 28.

### Example 1 Construction of Wheat Gene Editing Vector

An sgRNA (SEQ ID NO. 1) required by CRISPR/SpCas9 gene editing system is designed near 1216-1218 sites in coding region of a Glu-1Ax-null gene of the Fielder. Four base sequences paired with the BsaI restriction sites of the vector pEtRNA are respectively inserted to the single strand of the sgRNA to obtain sequences (5′-3′): tgcacttggctgctgctcttatcc (SEQ ID NO.30) and aaacggataagagcagcagccaag (SEQ ID NO.31), wherein the sequences of the underlined front four bases are the inserted sequences, and the two sequences are synthesized by Beijing Tsingke Biotechnology Co., Ltd. and form double-stranded sgRNA after annealing. The vector pEtRNA is digested by BsaI endonuclease, the digested vector is extracted by an agarose gel extraction kit, the digested vector and the annealed double-stranded sgRNA are ligated overnight by T4 ligase, and the ligated product is transformed into Escherichia coli.

Verifying whether the sgRNA is successfully cloned onto BsaI sites of the vector pEtRNA by sequencing with a primer pTaU3 (F: GTAAAACGACGGCCAGT, SEQ ID NO. 25; R: TGCACTGCAGGCATGCAA, SEQ ID NO. 26).

The vector of the Escherichia coli for positive clones is transformed into Agrobacterium EHA105 by chemical transformation, and a positive Agrobacterium clone is verified by PCR with the primer pTaU3 (F: GTAAAACGACGGCCAGT, SEQ ID NO. 25; R: TGCACTGCAGGCATGCAA, SEQ ID NO. 26), and positive clones are screened.

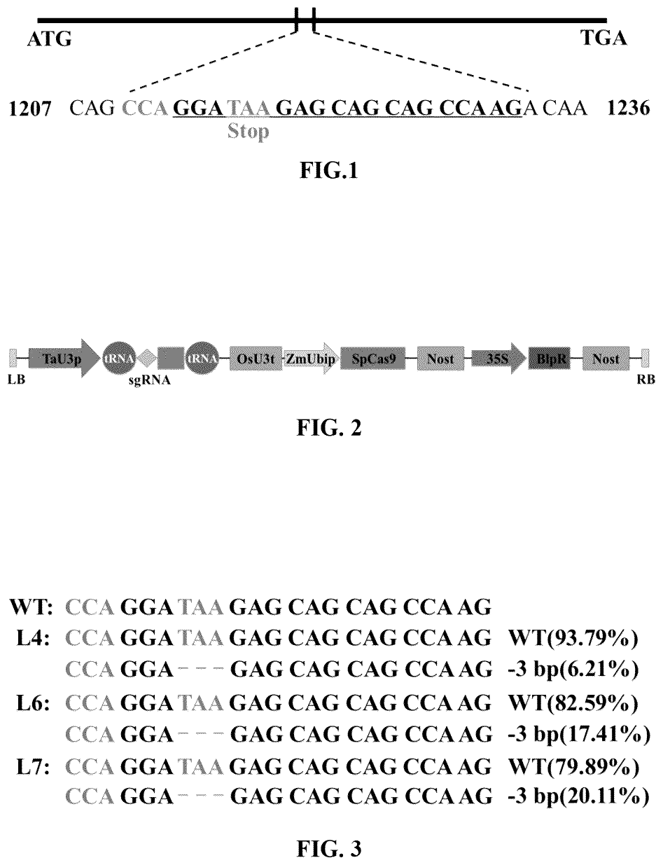

FIG. 1 is schematic diagram of the target sequence of the sgRNA as set forth in SEQ ID. 32 in Example 1, wherein the underlined sequence represents the target sequence of the sgRNA, the three bases separated by spaces represent amino acid codon, the three bases corresponding to Stop represent the premature termination codon, and the three bases (CCA) at the upstream of the underlined target sequence of the sgRNA represent PAM sequence. FIG. 2 shows a schematic diagram of the vector employed for gene editing as set forth in SEQ ID NO. 2 in examples of the present invention, wherein the diamond represents the sgRNA as set forth in SEQ ID NO. 1, and the square at its downstream represents the sgRNA scaffold sequence (i.e., the MSS sequence), wherein the MSS sequence is shown as SEQ ID NO. 21; and the tRNA is shown as SEQ ID NO. 19.

### Example 2 Construction of Transgenic Wheat Line and Genotyping

The gene editing vector constructed in Example 1 is transformed into wheat cell via Agrobacterium-mediated transformation method to construct transgenic wheat line.

(1) Whole genomic DNA of wheat leaves is extracted by CTAB method (Murray and Thompson, 1980). Amplification is performed to the target site region with the specific primer shown in Table 1. High-throughput sequencing is performed to PCR product by Hi-TOM sequencing method to identify the edition situation of the target site of T0-generation transgenic line (referring to Liu, Q., Wang, C., Jiao, X., Zhang, H., Song, L., Li, Y., Gao, C., Wang, K. (2019). Hi-TOM: a platform for high-throughput tracking of mutations induced by CRISPR/Cas systems. Science China. Life Sciences, 62(1), 1-7.). Lines with a high proportion of high-throughput sequencing edited reads are selected for greenhouse generation-adding while detecting the edited situation of the target site by the same method, and gene-edited homozygous lines are screened.

TABLE 1
PCR primer sequence required by
Hi-TOM sequencing

| Primer Name | pGlu |
|---|---|
| Forward Primer | GGAGTGAGTACGGTGTGCACAA |
| (5′-3′) | TTAGGACAAGGGCAACCAA |
| | SEQ ID NO. 22 |
| Reverse Primer | GAGTTGGATGCTGGATGGCTGG |
| (5′-3′) | CTGCTGACCTTGCTC |
| | SEQ ID NO. 23 |

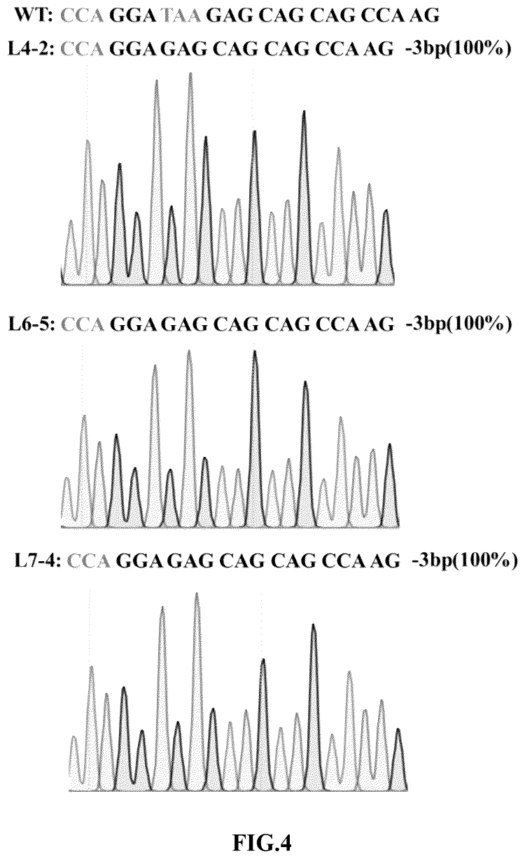

The gene editing vector is transformed into wheat cell via Agrobacterium-mediated transformation, and screening is performed to obtain 12 T0-generation transgenic lines. The genotypes of transgenic lines are verified by Hi-TOM sequencing method to find 3bp sequence deletion in three lines (marked as L4, L6 and L7, respectively) with proportions of the genotypes accounting for 6.21%, 17.41% and 20.11%, respectively, and the premature termination codon TAA is deleted, wherein the results are shown in FIG. 3. In FIG. 3, WT represents wild type as set forth in SEQ ID NO. 33, CCA represents PAM sequence, TAA represents the premature termination codon deleted by gene editing, the three bases separated by the spaces represent amino acid codon, short lines represent the bases deleted by gene editing (each short line represents one base), sequence of the genotypes in which the premature termination codon TAA is deleted is set forth in SEQ ID NO. 34, and the percentage in parentheses represents the proportion of Hi-TOM sequencing genotypes in reads.

(2) Greenhouse generation-adding is performed to T0-generation lines while verifying progeny genotypes, screening is performed to achieve that the proportion of −3bp genotype in a single plant reaches 100% in T4-generation. T4-generation L4-2, L6-5 and L7-4 lines shown in FIG. 4 are selected and their seeds are sown in a transgenic experimental field, and HMW-GS composition in seeds and flour processing quality are analyzed to verify gene editing effect. In FIG. 4, WT represents wild type as set forth in SEQ ID NO. 33, CCA represents PAM sequence, TAA represents the premature termination codon deleted by gene editing, the three bases separated by the spaces represent amino acid codon, sequence of the genotypes in which the premature termination codon TAA is deleted is set forth in SEQ ID NO. 34, and the percentage in parentheses represents the proportion of Hi-TOM sequencing genotypes in reads.

### Example 3 HMW-GS Composition Analysis and Verification

The gene-edited homozygous lines are sown to Jinan transgenic test field, Crop Research Institute, Shandong Academy of Agricultural Sciences. The field experiment is conducted in randomized complete blocks design with three replicates, with plot area of 6 square meters, 4-meter long, 6-row regions and 0.2-meter row spacing. The field management is carried out according to conventional management method. The grain protein content (14% moisture basis) and moisture are determined by near infrared analyzer (Foss 1241, Sweden), and the sample moisture is adjusted to 14% for wheat moistening while the wheat moistening time is 16-18 h. Flour is made by Quadrumat Junior mill (Brabender, Germany) and according to AACC 26-50 method and is sieved by a 100-mesh sieve for HMW-GS composition and processing quality analysis. The HMW-GS compositions of Fielder wild type and gene-edited line seeds are analyzed by method proposed by Gupta et al. (1993). The differentially expressed proteins verified by SDS-PAGE are verified by LC/MS. The mass spectrometry is performed by Beijing Novogene Biotech Co., Ltd.

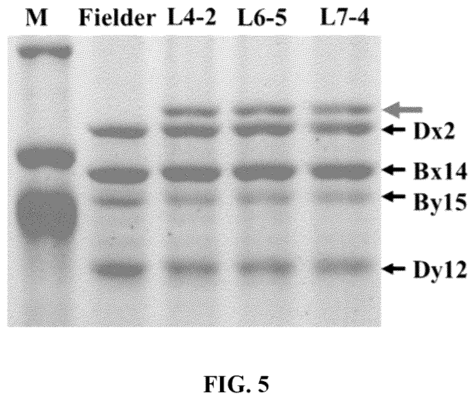

Seed HMW-GS are extracted, and the HMW-GS composition of the gene-edited line is analyzed by SDS-PAGE. As shown in FIG. 5, only 4 HMW-GS are expressed in Fielder wild type line, an extra protein band is shown on Dx2 subunit of each of the gene-edited lines L4-2, L6-5 and L7-4, and a total of 5 HMW-GS are expressed.

The protein bands on the Dx2 subunit in FIG. 5 are analyzed by mass spectrometry, and the protein with the most annotation content is Glu-1Ax subunit, which accounts for 97.1%, corresponds to protein sequence ID Q41516 in the Uniprot database and is described as wheat high-molecular-weight glutenin subunit. Its encoding gene has sequence ID U19774 in the GenBank database, which is described as wheat high-molecular-weight glutenin Glu-1Ax encoding gene and has identity up to 99.92% as Fielder Glu-1Ax null gene sequence. Therefore, the protein bands on the Dx2 subunits of lines L4-2, L6-5 and L7-4 are the activated Glu-1Ax-null subunits.

### Example 4 Wheat Processing Quality Analysis

The Zeleny settlement value (ZSV) of flour is determined by AACC 56-63 method with result corrected to 14% moisture content. Wet gluten content is measured by 2200-type gluten quantity and quality measurement system (Perten, Sweden) and according to GB/T 5506.2-2008 method, and gluten index (GI), i.e., gluten retained on the gluten sieve/(gluten retained on the gluten sieve+sieved gluten)×100, is determined by a gluten sieve in Perten Centrifuge 2015. Dough stability time (ST) and flour quality number (FQN) are measured by a farinograph (Brabender, Germany) and according to AAC-54-21 method. Each sample is repeatedly measured for 3 times. SPSS22 software (https://www.ibm.com/analytics/data-science/pre) is applied for data statistical analysis. Multiple comparisons between the data are performed by LSD method with 0.05 as significant level.

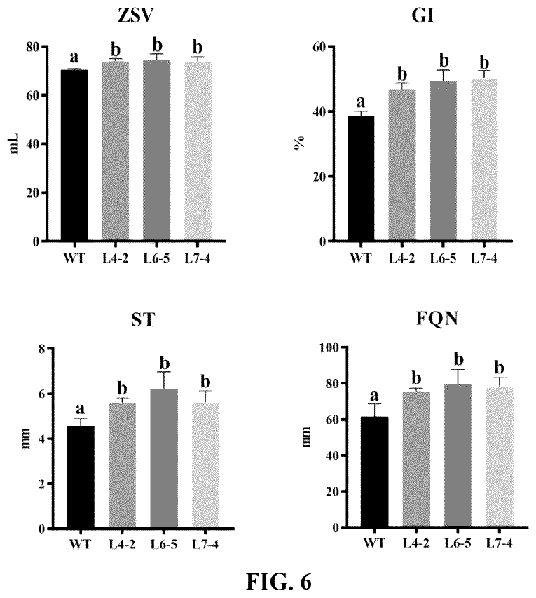

According to the method, flour processing qualities of Fielder wild type line and gene-edited lines are analyzed, respectively, and the results are shown in FIG. 6. The data in FIG. 6 shows that, compared with the wild type line, the flour settlement value (ZSV), gluten index (GI), stability time (ST) and flour quality number (FQN) of gene-edited lines are significantly improved by about 5.2-6.1%, 21.5-31.4%, 22.5-36.5% and 22.2-29.2%, respectively. The results of the example indicate that activation of the silenced Glu-1Ax-null subunit in Fielder can improve wheat processing quality.

### Comparative Examples

Before determining the gene editing method of the present invention and related biological materials, the inventor of the present invention conducted a large number of experiments. The following comparative examples specify some experimental schemes that failed to activate expression of a Glu-1Ax-null subunit in wheat.

### Comparative Examples 1-8

Wheat gene editing vector is constructed, transgenic wheat lines are obtained and genotype analysis is performed in comparative examples 1-8 by using the same gene editing tool SpCas9 as the present invention and according to the methods in examples 1-2 except that the selected sgRNA and/or the adopted gene editing vectors are different. The specific vector information, sgRNA sequences and gene editing results are as follows.

### Comparative Example 1

sgRNA sequence: ggagaagttgggtagtacct (SEQ ID NO. 4)

A vector comprises a U3-sgRNA expression cassette regulated by a wheat TaU3 promoter and an SpCas9 expression cassette regulated by a ZmUbi promoter, wherein the U3-sgRNA expression cassette includes, from upstream to downstream: the wheat TaU3 promoter, an sgRNA+non-optimized sgRNA scaffold sequence for targeting encoding 1183-1202 region (upstream of premature termination codon) in a Glu-1Ax-null gene of wheat, and an OsU3 terminator (non-optimized sgRNA scaffold+conventional sgRNA release manner).

Objective: to create a mutation at upstream of the premature termination codon, causing a frameshift mutation and disrupting the premature termination codon.

Results: no edition.

### Comparative Example 2

sgRNA sequence:
(SEQ ID NO. 1)
cttggctgctgctcttatcc

Vector information: a vector in the comparative example 2 comprises a U3-sgRNA expression cassette regulated by a wheat TaU3 promoter and an SpCas9 expression cassette regulated by a ZmUbi promoter, wherein the U3-sgRNA expression cassette includes, from upstream to downstream: the wheat TaU3 promoter, a specific sgRNA+non-optimized sgRNA scaffold sequence for targeting the premature termination codon in a Glu-1Ax-null gene of wheat, and an OsU3 terminator (non-optimized sgRNA scaffold+conventional sgRNA release manner).

Objective: to create a mutation at the premature termination codon, causing a frameshift mutation and disrupting the premature termination codon.

Results: insertion of a base C in front of the premature termination codon TAA causes frameshift mutation, but protein is still not expressed.

### Comparative Example 3

sgRNA sequence:
(SEQ ID NO. 5)
ctccgcaacaattaggacaa

Vector information: a vector in the comparative example 3 comprises a U3-sgRNA expression cassette regulated by a wheat TaU3 promoter and an SpCas9 expression cassette regulated by a ZmUbi promoter, wherein the U3-sgRNA expression cassette includes, from upstream to downstream: the wheat TaU3 promoter, an sgRNA+non-optimized sgRNA scaffold sequence for targeting encoding 1154-1173 region (upstream of premature termination codon) in a Glu-1Ax-null gene of wheat, and an OsU3 terminator (non-optimized sgRNA scaffold+conventional sgRNA release manner).

Objective: to create a mutation at the upstream of the premature termination codon, causing a frameshift mutation and disrupting the premature termination codon.

Results: no edition.

### Comparative Example 4

sgRNA sequence:
(SEQ ID NO. 4)
ggagaagttgggtagtacct

Vector information: a vector in the comparative example 4 comprises a U3-sgRNA expression cassette regulated by a wheat TaU3 promoter and an SpCas9 expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1183-1202 region (upstream of premature termination codon) in a Glu-1Ax-null gene of wheat, a second tRNA, and an OsU3 terminator (optimized sgRNA scaffold+tRNA-mediated sgRNA release manner).

Objective: to create a mutation at upstream of the premature termination codon, causing a frameshift mutation and disrupting the premature termination codon.

Results: no edition.

### Comparative Example 5

sgRNA sequence:
(SEQ ID NO. 6)
ctggctgctgcggagaagtt

Vector information: a vector in the comparative example 5 comprises a U3-sgRNA expression cassette regulated by a wheat TaU3 promoter and an SpCas9 expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1194-1213 region (upstream of premature termination codon) in a Glu-1Ax-null gene of wheat, a second tRNA, and an OsU3 terminator (optimized sgRNA scaffold+tRNA-mediated sgRNA release manner).

Objective: to create a mutation at the upstream of the premature termination codon, causing a frameshift mutation and disrupting the premature termination codon.

Results: no edition.

### Comparative Example 6

sgRNA sequence:
(SEQ ID NO. 7)
tgctcttatcctggctgctg

Vector information: a vector in the comparative example 6 comprises a U3-sgRNA expression cassette regulated by a wheat TaU3 promoter and an SpCas9 expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1204-1223 region (upstream of premature termination codon) in a Glu-1Ax-null gene of wheat, a second tRNA, and an OsU3 terminator (optimized sgRNA scaffold+tRNA-mediated sgRNA release manner).

Objective: to create a mutation at the upstream of the premature termination codon, causing a frameshift mutation and disrupting the premature termination codon.

Results: no edition.

### Comparative Example 7

sgRNA sequence:
(SEQ ID NO. 5)
ctccgcaacaattaggacaa

Vector information: a vector in the comparative example 7 comprises a U3-sgRNA expression cassette regulated by a wheat TaU3 promoter and an SpCas9 expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1154-1173 region (upstream of premature termination codon) in a Glu-1Ax-null gene of wheat, a second tRNA, and an OsU3 terminator (optimized sgRNA scaffold+tRNA-mediated sgRNA release manner).

Objective: to create a mutation at upstream of the premature termination codon, causing a frameshift mutation and disrupting the premature termination codon.

Results: no edition.

### Comparative Example 8

sgRNA sequence:
(SEQ ID NO. 8)
ccaacttctccgcagcagcc

Vector information: a vector in the comparative example 8 comprises a U3-sgRNA expression cassette regulated by a wheat TaU3 promoter and an SpCas9 expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1192-1221 region (upstream of premature termination codon) in a Glu-1Ax-null gene of wheat, a second tRNA, and an OsU3 terminator (optimized sgRNA scaffold+tRNA-mediated sgRNA release manner).

Objective: to create a mutation at the upstream of the premature termination codon, causing a frameshift mutation and disrupting the premature termination codon.

Results: no edition.

### Comparative Examples 9-14

Wheat gene editing vector is constructed, transgenic wheat lines are obtained and genotype analysis is performed in comparative examples 9-14 by using another common gene editing tool SpCas9-NG different from that in the present invention and according to the methods in examples 1-2 except that the selected sgRNA and/or the adopted gene editing vectors are different. The specific vector information, sgRNA sequences and gene editing results are as follows.

### Comparative Example 9

sgRNA sequence:
(SEQ ID NO. 4)
ggagaagttgggtagtacct

Vector information: a vector in the comparative example 9 comprises a U3-sgRNA expression cassette regulated by a wheat TaU3 promoter and an SpCas9-NG expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1183-1202 region (upstream of premature termination codon) in a Glu-1Ax-null gene of wheat, a second tRNA, and an OsU3 terminator (optimized sgRNA scaffold+tRNA-mediated sgRNA release manner).

Objective: to create a mutation at the upstream of the premature termination codon, causing a frameshift mutation and disrupting the premature termination codon.

Results: no edition.

### Comparative Example 10

sgRNA sequence:
(SEQ ID NO. 9)
ctacccaacttctccgcagc

Vector information: a vector in the comparative example 10 comprises a U3-sgRNA expression cassette regulated by a wheat TaU3 promoter and an SpCas9-NG expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1188-1207 region (upstream of premature termination codon) in a Glu-1Ax-null gene of wheat, a second tRNA, and an OsU3 terminator (optimized sgRNA scaffold+tRNA-mediated sgRNA release manner).

Objective: to create a mutation at the upstream of the premature termination codon, causing a frameshift mutation and disrupting the premature termination codon.

Results: no edition.

### Comparative Example 11

sgRNA sequence:
(SEQ ID NO. 10)
tatcctggctgctgcggaga

Vector information: a vector in the comparative example 11 comprises a U3-sgRNA expression cassette regulated by a wheat TaU3 promoter and an SpCas9-NG expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1198-1217 region (upstream of premature termination codon) in a Glu-1Ax-null gene of wheat, a second tRNA, and an OsU3 terminator (optimized sgRNA scaffold+tRNA-mediated sgRNA release manner).

Objective: to create a mutation at the upstream of the premature termination codon, causing a frameshift mutation and disrupting the premature termination codon.

Results: no edition.

### Comparative Example 12

sgRNA sequence:
(SEQ ID NO. 1)
cttggctgctgctcttatcc

Vector information: the vector in the comparative example 12 comprises a U3-sgRNA expression cassette regulated by a wheat TaU3 promoter and an SpCas9-NG expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1213-1232 premature termination codon region in a Glu-1Ax-null gene of wheat, a second tRNA, and an OsU3 terminator (optimized sgRNA scaffold+tRNA-mediated sgRNA release manner).

Objective: to create a mutation at the premature termination codon, causing a frameshift mutation and disrupting the premature termination codon.

Results: no edition.

### Comparative Example 13

sgRNA sequence:
(SEQ ID NO. 11)
tccgcagcagccaggataag

Vector information: a vector in the comparative example 13 comprises a U3-sgRNA expression cassette regulated by a wheat TaU3 promoter and an SpCas9-NG expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1200-1219 premature termination codon region in a Glu-1Ax-null gene of wheat, a second tRNA, and an OsU3 terminator (optimized sgRNA scaffold+tRNA-mediated sgRNA release manner).

Objective: to create a mutation at the premature termination codon, causing a frameshift mutation and disrupting the premature termination codon.

Results: no edition.

### Comparative Example 14

sgRNA sequence:
(SEQ ID NO. 12)
gcagcagccaggataagagc

Vector information: a vector in the comparative example 14 comprises a U3-sgRNA expression cassette regulated by a wheat TaU3 promoter and an SpCas9-NG expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1203-1222 premature termination codon region in a Glu-1Ax-null gene of wheat, a second tRNA, and an OsU3 terminator (optimized sgRNA scaffold+tRNA-mediated sgRNA release manner).

Objective: to create a mutation at the premature termination codon, causing a frameshift mutation and disrupting the premature termination codon.

Results: no edition.

### Comparative Examples 15-19

Wheat gene editing vector is constructed, transgenic wheat lines are obtained and genotype analysis is performed in comparative examples 15-19 by using a common gene editing tool SpRY different from that in the present invention and according to the methods in examples 1-2 except that the selected sgRNA and/or the adopted gene editing vectors are different. The specific vector information, sgRNA sequences and gene editing results are as follows.

### Comparative Example 15

sgRNA sequence:
(SEQ ID NO. 13)
aggtactacccaacttctcc

Vector information: a vector in the comparative example 15 comprises a U3-sgRNA expression cassette regulated by a wheat TaU3 promoter and an SpRY expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1183-1202 region (upstream of premature termination codon) in a Glu-1Ax-null gene of wheat, a second tRNA, and an OsU3 terminator (optimized sgRNA scaffold+tRNA-mediated sgRNA release manner).

Objective: to create a mutation at the upstream of the premature termination codon, causing a frameshift mutation and disrupting the premature termination codon.

Results: no edition.

### Comparative Example 16

sgRNA sequence:
(SEQ ID NO. 14)
caacttctccgcagcagcca

Vector information: a vector in the comparative example 16 comprises a U3-sgRNA expression cassette regulated by a wheat TaU3 promoter and an SpRY expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1193-1212 region (upstream of premature termination codon) in a Glu-1Ax-null gene of wheat, a second tRNA, and an OsU3 terminator (optimized sgRNA scaffold+tRNA-mediated sgRNA release manner).

Objective: to create a mutation at the upstream of the premature termination codon, causing a frameshift mutation and disrupting the premature termination codon.

Results: no edition.

### Comparative Example 17

sgRNA sequence:
(SEQ ID NO. 1)
cttggctgctgctcttatcc

Vector information: a vector in the comparative example 17 comprises a U3-sgRNA expression cassette regulated by a wheat TaU3 promoter and an SpRY expression cassette regulated by a ZmUbi promoter, wherein the U3-sgRNA expression cassette includes, from upstream to downstream: the wheat TaU3 promoter, a first tRNA, a specific sgRNA+MSS sequence for targeting encoding 1213-1232 premature termination codon region in a Glu-1Ax-null gene of wheat, a second tRNA, and an OsU3 terminator (optimized sgRNA scaffold+tRNA-mediated sgRNA release manner).

Objective: to create a mutation at the premature termination codon, causing a frameshift mutation and disrupting the premature termination codon.

Results: no edition.

### Comparative Example 18

sgRNA sequence:
(SEQ ID NO. 15)
cgcagcagccaggataagag

Vector information: a vector in the comparative example 18 comprises a U3-sgRNA expression cassette regulated by a wheat TaU3 promoter and an SpRY expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1202-1221 premature termination codon region in a Glu-1Ax-null gene of wheat, a second tRNA, and an OsU3 terminator (optimized sgRNA scaffold+tRNA-mediated sgRNA release manner).

Objective: to create a mutation at the premature termination codon, causing a frameshift mutation and disrupting the premature termination codon.

Results: no edition.

### Comparative Example 19

sgRNA sequence:
(SEQ ID NO. 16)
ccgcagcagccaggataaga

Vector information: s vector in the comparative example 19 comprises a U3-sgRNA expression cassette regulated by a wheat TaU3 promoter and an SpRY expression cassette regulated by a ZmUbi promoter, wherein the U3-sgRNA expression cassette includes, from upstream to downstream: the wheat TaU3 promoter, a first tRNA, an sgRNA+MSS sequence for targeting encoding 1201-1220 premature termination codon region in a Glu-1Ax-null gene of wheat, a second tRNA, and an OsU3 terminator (optimized sgRNA scaffold+tRNA-mediated sgRNA release manner).

Objective: to create a mutation at the premature termination codon, causing a frameshift mutation and disrupting the premature termination codon.

Results: no edition.

### Comparative Examples 20-21

Wheat gene editing vector is constructed, transgenic wheat lines are obtained and genotype analysis is performed in comparative examples 20-21 by using a common gene editing tool ABE9-SpRY different from that in the present invention and according to the methods in examples 1-2 except that the selected sgRNA and/or the adopted gene editing vectors are different. The specific vector information, sgRNA sequences and gene editing results are as follows.

### Comparative Example 20

sgRNA sequence:
(SEQ ID NO. 17)
tcttatcctggctgctgcgg

Vector information: a vector in the comparative example 20 comprises a U3-sgRNA expression cassette regulated by a wheat TaU3 promoter and an ABE9-SpRY expression cassette regulated by a ZmUbi promoter, wherein the U3-sgRNA expression cassette includes, from upstream to downstream: the wheat TaU3 promoter, a first tRNA, an sgRNA+MSS sequence for targeting encoding region 1216-site base T in a Glu-1Ax-null gene of wheat, a second tRNA, and an OsU3 terminator (optimized sgRNA scaffold+tRNA-mediated sgRNA release manner).

Objective: to create a mutation at the premature termination codon, converting TAA into GAA and disrupting the premature termination codon.

Results: no edition.

### Comparative Example 21

sgRNA sequence:
(SEQ ID NO. 18)
gataagagcagcagccaaga

Vector information: a vector in the comparative example 21 comprises a U3-sgRNA expression cassette regulated by a wheat TaU3 promoter and an ABE9-SpRY expression cassette regulated by a ZmUbi promoter, wherein the U3-sgRNA expression cassette includes, from upstream to downstream: the wheat TaU3 promoter, a first tRNA, an sgRNA+MSS sequence for targeting encoding region 1,217-site and 1,218-site base A in a Glu-1Ax-null gene of wheat, a second tRNA, and an OsU3 terminator (optimized sgRNA scaffold+tRNA-mediated sgRNA release manner).

Objective: to create a mutation at the premature termination codon, converting TAA into TGG and disrupting the premature termination codon.

Results: no edition.

What is claimed is:

1. A method for activating expression of a Glu-1Ax-null subunit in wheat with gene editing, comprising the steps of:
designing a specific sgRNA for targeting a premature termination codon in a Glu-1Ax-null gene of wheat;
cloning the specific sgRNA onto a pair of BsaI sites of a vector pEtRNA to construct a CRISPR/SpCas9 editing vector for the Glu-1Ax-null gene; and
transforming the CRISPR/SpCas9 editing vector for the Glu-1Ax-null gene into the wheat cell via Agrobacterium-mediated transformation and selecting a homozygous line in which the premature termination codon is disrupted, wherein:
a sequence of the vector pEtRNA is shown as SEQ ID NO. 3.

2. The method in claim 1, wherein a target sequence of the specific sgRNA corresponds to a sequence having a length of 20-22 nucleotides between the 1208-1235 sites of the Glu-1Ax-null gene.

3. The method in claim 1, wherein a nucleotide sequence of the specific sgRNA is shown as SEQ ID NO. 1.

4. The method in claim 1, wherein:
the CRISPR/SpCas9 editing vector for the Glu-1Ax-null gene includes:
a U3-sgRNA expression cassette regulated by a wheat TaU3 promoter; and
an SpCas9 expression cassette regulated by a ZmUbi promoter;
the U3-sgRNA expression cassette includes, from upstream to downstream:
the wheat TaU3 promoter;
a first tRNA;
the specific sgRNA for targeting the premature termination codon in the Glu-1Ax-null gene of wheat;
an MSS sequence;
a second tRNA which is identical to the first tRNA; and
an OsU3 terminator; and
the MSS sequence is shown as SEQ ID NO. 21.

5. The method in claim 1, wherein:
the first tRNA is shown as SEQ ID NO. 19; and
the second tRNA is shown as SEQ ID NO. 19

6. The method in claim 1, wherein a nucleotide sequence of the CRISPR/SpCas9 editing vector for the Glu-1Ax-null gene is shown as SEQ ID NO. 2.

7. The method in claim 1, further comprising the steps of:
transforming the CRISPR/SpCas9 editing vector for the Glu-1Ax-null gene which contains the specific sgRNA and an SpCas9 expression element into Escherichia coli; screening the Escherichia coli for positive clones; and verifying the positive clones through sequencing;
transforming a vector of the Escherichia coli into Agrobacterium through chemical transformation; using PCR to identify a positive Agrobacterium clone; and introducing the positive Agrobacterium clone into wheat to obtain transgenic wheat; and
screening the transgenic wheat for a gene-edited plant in which the premature termination codon is disrupted; self-crossing the plant to obtain a progeny; and selecting the homozygous line from the progeny in which the premature termination codon is disrupted.

8. A specific sgRNA for targeting a premature termination codon in a Glu-1Ax-null gene of wheat, wherein a target sequence of the specific sgRNA corresponds to a sequence having a length of 20-22 nucleotides between the 1208-1235 sites of the Glu-1Ax-null gene.

9. The specific sgRNA in claim 8, wherein a nucleotide sequence of the specific sgRNA is shown as SEQ ID NO. 1.

10. A CRISPR/SpCas9 editing vector for a Glu-1Ax-null gene of wheat, comprising:
a U3-sgRNA expression cassette regulated by a wheat TaU3 promoter; and
an SpCas9 expression cassette regulated by a ZmUbi promoter, wherein:
the U3-sgRNA expression cassette includes, from upstream to downstream:
the wheat TaU3 promoter;
a first tRNA;
the specific sgRNA for targeting the premature termination codon in the Glu-1Ax-null gene of wheat;
an MSS sequence;
a second tRNA which is identical to first tRNA; and
an OsU3 terminator; and
the MSS sequence is shown as SEQ ID NO. 21.

11. The CRISPR/SpCas9 editing vector in claim 10, wherein:
the first tRNA is shown as SEQ ID NO. 19; and
the second tRNA is shown as SEQ ID NO. 19.

12. The CRISPR/SpCas9 editing vector in claim 10, wherein a nucleotide sequence of the CRISPR/SpCas9 editing vector for the Glu-1Ax-null gene is shown as SEQ ID NO. 2.
